(19) United States
(12) Patent Application Publication
(10) Pub. No.: US 2026/0315093 A1
(43) Pub. Date: Oct. 15, 2026

(54) ULTRASOUND INTERROGATION FOR RENAL DENERVATION

(71) Applicant: Boston Scientific Scimed, Inc., Maple Grove, MN (US)

(72) Inventors: Corydon Carlson, Stillwater, MN (US); Wenguang Li, Los Gatos, CA (US)

(73) Assignee: Boston Scientific Scimed, Inc., Maple Grove, MN (US)

(21) Appl. No.: 19/463,290

(22) Filed: Jan. 29, 2026

Related U.S. Application Data
(60) Provisional application No. 63/750,999, filed on Jan. 29, 2025.

(51) Int. Cl. A61B 17/32; A61B 8/12; A61B 17/00

(52) U.S. Cl. CPC A61B 17/320068; A61B 8/12; A61B 2017/00238; A61B 2017/00292; A61B 2017/320069

(57) ABSTRACT

The disclosure provides a renal denervation system arranged to modulate (or change) ablation energy based on indications of intraluminal structure in a renal artery. The renal denervation system can include a combined imaging and renal denervation catheter arranged to both interrogate the renal artery (e.g., capture images of, capture indications of intraluminal structure) and to emit ablation energy.

### CROSS-REFERENCE TO RELATED APPLICATION

This application claims the benefit of U.S. Patent Application Serial No. 63/750,999, filed Jan. 29, 2025, entitled " ULTRASOUND INTERROGATION FOR RENAL DENERVATION”, which is incorporated by reference herein in its entirety.

### TECHNICAL FIELD

The present disclosure generally relates to medical denervation or ablation systems and particularly, but not exclusively, to renal denervation and interrogating the area to be treated with ultrasound.

### BACKGROUND

Hypertension is the leading risk factor for cardiovascular disease in the United States and globally, and it has been well established that treatment of hypertension reduces cardiovascular morbidity and mortality and slows progression to end-stage kidney disease. Despite treatment with lifestyle modifications, antihypertensive medications, or both, blood pressure (BP) control rates remain poor. As of 2014, there were nearly 120 million adults in the United States with hypertension. Of those, only 23% have controlled BP to the recommended target of <130/80 mm Hg, and ≈45% of those with uncontrolled hypertension have BP readings of ≥140/90 mm Hg. (Bromfield, Samantha G., et al. "Trends in hypertension prevalence, awareness, treatment, and control among US adults 80 years and older, 1988–2010." The Journal of Clinical Hypertension 16.4 (2014): 270-276.)

It is understood that sympathetic signaling between the central nervous system and the kidneys plays a role in the maintenance of high BP. Renal denervation (RDN) is a minimally invasive procedure that targets this sympathetic signaling and is a treatment option for patients with uncontrolled or treatment resistant hypertension. RDN operates by causing neuromodulation of the sympathetic signaling between the central nervous system and the kidneys via denervation of the renal nerves. In general, during the procedure an interventional physician uses a catheter inserted into the renal arteries (e.g., via the femoral artery, or the like) to apply energy to the renal arteries and surrounding tissue.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

As introduced above, RDN aims to reduce hypertension via neuromodulation of the renal arteries. There are a variety of energy modalities that can be used for RDN, such as, for example, ultrasound, radio frequency (RF), pulsed electric field (PEF), or the like. However, an issue with current RDN energy modalities is there are often structures in the artery that interfere with, or otherwise limit, the intended ablation energy reaching the target (e.g., the renal nerves).

For example, lesions (e.g., calcium, plaque, stenosed regions, or the like) in the artery may interfere with application of ablation energy. Thus, there is a need for medical devices, medical systems, and/or medical techniques to identify and/or avoid areas in the artery, to apply ablation energy based on the artery structure, or to both identify areas of interference in the artery and/or to selectively apply ablation energy based on the identified areas.

In general, the disclosure provides an intraluminal ultrasound catheter that is sized for insertion into the renal arteries and configured with one or more rotatable ultrasound transducers on its distal end. The one or more rotatable ultrasound transducers and the intraluminal ultrasound catheter can be configured to capture images from within the artery prior to (or as part of) an RDN procedure. As an example, lesions within the artery can be identified and/or mapped from the captured images. Subsequently, the RDN procedure can be implemented based on the identified lesion or the lesion mapping. For example, areas that may interfere with the ablation energy (e.g., calcium thicker than a threshold amount, or the like) can be avoided, or alternatively ablation energy can be increased in these areas to compensate for the interference.

With some embodiments, the intraluminal ultrasound catheter can be combined with the ablation catheter while in other embodiments, the intraluminal ultrasound catheter can be separate from the ablation catheter.

In some embodiments, the disclosure can be implemented as a renal denervation (RDN) system. The RDN system can comprise a generator configured to generate ablation activation signals to cause an RDN catheter to emit ablation energy; a processor coupled to the generator; and a memory comprising instructions, which when executed by the processor cause the processor to receive, from an image acquisition device, indications of intraluminal structure; generate, via the generator, the ablation activation signals based on the indications of the intraluminal structure; and communicate the ablation activation signals to the RDN catheter.

With further embodiments, the RDN system can comprise the RDN catheter.

With further embodiments of the RDN system, the RDN catheter comprises an elongate member; a drive shaft disposed within the elongate member; and a plurality of ultrasound transducers disposed on a distal end of the drive shaft and configured to emit the ablation energy in the form of acoustic energy.

With further embodiments of the RDN system, the plurality of ultrasound transducers are spaced linearly along the driveshaft.

With further embodiments of the RDN system, each of the plurality of ultrasound transducers are spaced a distance from adjacent ones of the plurality of ultrasound transducers, and wherein the distance is between 0.2 millimeters (mm) and 5.0 mm or wherein the distance is between 0.5 mm and 2.0 mm.

With further embodiments of the RDN system, the plurality of ultrasound transducers comprise between one (1) and eleven (11). ultrasound transducers

With further embodiments, the RDN system can comprise the image acquisition device.

With further embodiments of the RDN system, the generator is a first generator, the processor is a first processor, and the memory is a first memory, and wherein the image acquisition device comprises a second generator configured to generate image activation signals to cause an imaging catheter to interrogate a renal artery; a second processor coupled to the second generator; and a second memory comprising instructions, which when executed by the second processor cause the second processor to generate, via the second generator, the image activation signals; communicate the image activation signals to the imaging catheter; receive, from the imaging catheter, the indications of the intraluminal structure; and communicate the indications of the intraluminal structure to the first processor.

With further embodiments of the RDN system, the imaging catheter and the RDN catheter are the same catheter.

With further embodiments, the RDN system can comprise a motor drive unit (MDU) coupled between the generator and the imaging catheter and/or the RDN catheter.

With further embodiments of the RDN system, the memory further comprises instructions that when executed by the processor cause the processor to modulate the ablation activation signals based on the indications of intraluminal structure, wherein the modulated ablation activation signals cause the RDN catheter to emit modulated ablation energy.

With further embodiments of the RDN system, the memory further comprises instructions that when executed by the processor cause the processor to generate a mapping of the intraluminal structure from the indications of the intraluminal structure; and modulate the ablation activation signals based on the mapping of the intraluminal structure, wherein the modulated ablation activation signals cause the RDN catheter to emit modulated ablation energy.

With further embodiments of the RDN system, the indications of intraluminal structure comprise indications of a lesion.

With further embodiments of the RDN system, the mapping comprises an indication of a location of the lesion along a longitudinal axis of the renal artery and wherein the ablation activation signals are modulated at the locations.

With further embodiments of the RDN system, the modulated ablation energy has a different power, different frequency, and/or a different pulse delay than the ablation energy.

In some embodiments, the disclosure can be implemented as a renal denervation (RDN) method. The RDN method can comprise providing an image acquisition device and an RDN device; positioning the image acquisition device and the RDN device in a location in a renal artery of a patient; and exposing, via the RDN device, the location in the renal artery to ablation energy to cause renal denervation of the renal nerves adjacent to the location in the renal artery, wherein the ablation energy is generated by a generator coupled to the image acquisition device and the RDN device, and wherein the ablation energy is generated based in part on indications, received from the image acquisition device, of intraluminal structure of the location in the renal artery.

With further embodiments of the RDN method, the image acquisition device and the RDN device are incorporated in an intravenous catheter.

With further embodiments, the RDN method can comprise moving the intravenous catheter to a secondary location in the renal artery; and exposing, via the RDN device, the secondary location in the renal artery to secondary ablation energy to cause renal denervation of the renal nerves adjacent to the secondary location in the renal artery, wherein the secondary ablation energy is generated based in part on indications, received from the image acquisition device, of intraluminal structure of the secondary location in the renal artery, and wherein the secondary ablation energy is different from the ablation energy.

In some embodiments, the disclosure can be implemented as a computer-readable storage device. The computer-readable storage device can be for a renal denervation (RDN) system. The computer-readable storage device can store instructions that when executed by a processor of a renal denervation (RDN) system, cause the RDN system to receive, from an image acquisition device coupled to the RDN system, indications of intraluminal structure of a renal artery; generate, via a generator of the RDN system, ablation activation signals based on the indications of the intraluminal structure; and communicate the ablation activation signals to an RDN catheter coupled to the RDN system, wherein the RDN catheter is configured to emit ablation energy responsive to the ablation activation signals.

With further embodiments of the computer-readable storage medium, the instructions when executed by the processor of the RDN system, further cause the RDN system to modulate the ablation activation signals based on the indications of intraluminal structure, wherein the modulated ablation activation signals cause the RDN catheter to emit modulated ablation energy.

With further embodiments of the computer-readable storage medium, the instructions when executed by the processor of the RDN system, further cause the RDN system to generate a mapping of the intraluminal structure from the indications of the intraluminal structure; and modulate the ablation activation signals based on the mapping of the intraluminal structure, wherein the modulated ablation activation signals cause the RDN catheter to emit modulated ablation energy.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an RDN system, in accordance with at least one embodiment.

FIG. 2 illustrates an intraluminal image acquisition system, in accordance with at least one embodiment.

FIG. 3 illustrates another RDN system, in accordance with at least one embodiment.

FIG. 4A illustrates yet another RDN system, in accordance with at least one embodiment.

FIGS. 4B and 4C illustrate a combined imaging and RDN catheter of the RDN system of FIG. 4A in greater detail.

FIG. 5A illustrates an example of human renal anatomy showing a renal artery.

FIG. 5B illustrates an example of a renal artery of the human renal anatomy of FIG. 5A in greater detail.

FIG. 5C illustrates an example of the human renal anatomy with the combined imaging and RDN catheter of FIGS. 4A, 4B, and 4C inserted into the renal artery of the human renal anatomy of FIGS. 5A and 5B.

FIG. 6 illustrates a logic flow to modulate ablation energy based on intraluminal structure, in accordance with at least one embodiment.

FIGS. 7A and 7B illustrate another logic flow to modulate ablation energy based on intraluminal structure, in accordance with at least one embodiment.

FIG. 8 illustrates a computer-readable storage medium, in accordance with at least one embodiment.

FIG. 9 illustrates a diagrammatic representation of a machine in the form of a computer system within which a set of instructions may be executed for causing the machine to perform any of the methodologies discussed herein, in accordance with at least one embodiment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its structure, organization, and operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

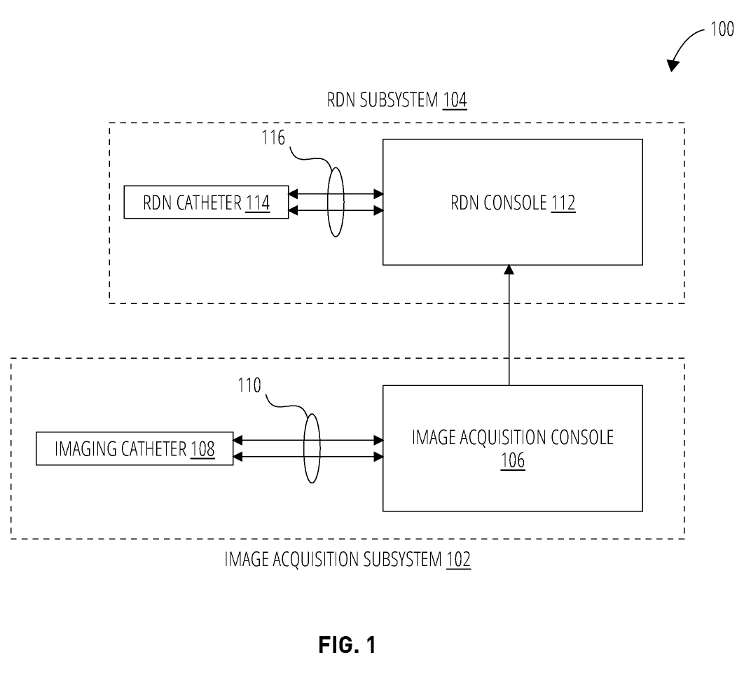

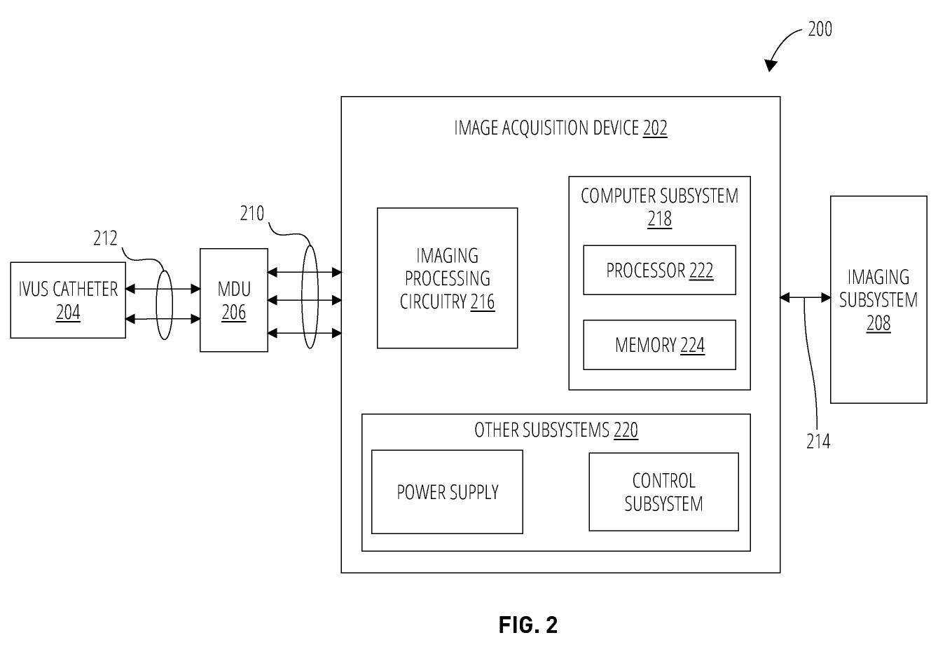

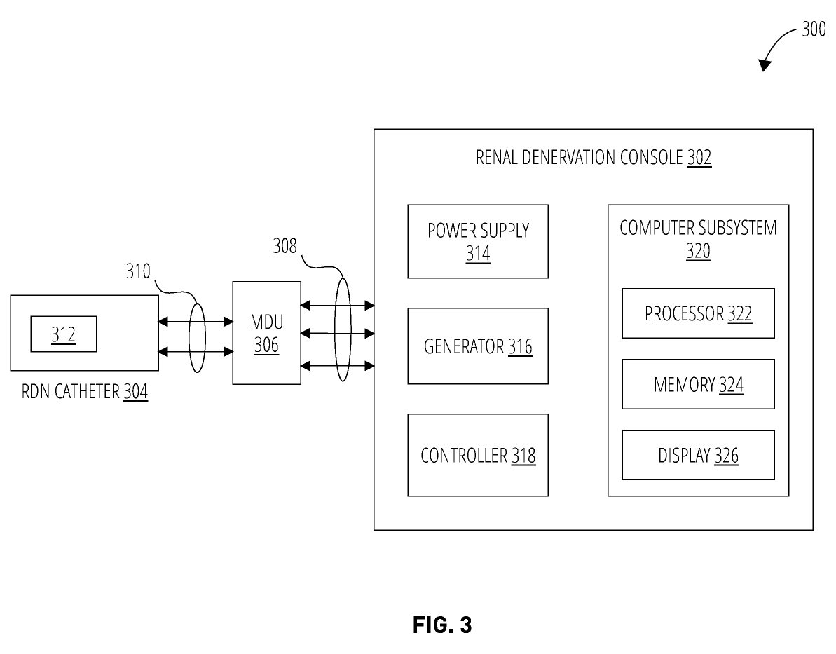

FIG. 1 illustrates an example renal denervation (RDN) system 100, according to at least one embodiment of the disclosure. RDN system 100 includes an image acquisition subsystem 102 and RDN subsystem 104. It is noted that although components of the image acquisition subsystem 102 and RDN subsystem 104 are depicted separately in this figure, some embodiments may be provided where components are combined. Example embodiments where some of these components are combined (e.g., the imaging and RDN catheters) are provided later in the disclosure. However, these subsystems are shown separately in this figure to provide clarity in describing their structure and function. Additionally, examples of each of the image acquisition subsystem 102 and RDN subsystem 104 are provided in FIGS. 2 and 3, respectively.

In general, the image acquisition subsystem 102 and RDN subsystem 104 are configured for use in imaging renal arteries and applying energy to renal nerves. To that end, the image acquisition subsystem 102 includes an image acquisition console 106 and an imaging catheter 108. The image acquisition console 106 and the imaging catheter 108 are coupled via an imaging catheter bus 110. In some embodiments, the imaging catheter bus 110 can be transmission lines (or other conductors) arranged to convey signals between the connected components. For example, the imaging catheter bus 110 can be arranged to transmit voltage signals (e.g., activation signals, control signals, ultrasound pulse generation signals, ultrasound echo return signals, or the like) between the connected components of the image acquisition subsystem 102. As another example, the imaging catheter bus 110 can be a digital signaling bus, an optical signaling bus, or the like.

Similarly, the RDN subsystem 104 includes an RDN console 112 and an RDN catheter 114. The RDN console 112 is coupled to RDN catheter 114 via the RDN catheter bus 116. Like the imaging catheter bus 110, the RDN catheter bus 116 can be transmission lines (or other conductors) arranged to convey signals between the connected components. For example, the RDN catheter bus 116 can be arranged to transmit voltage signals (e.g., activation signals, control signals, ablation energy signals, or the like) from the RDN console 112 to the RDN catheter 114.

In general, the imaging catheter 108 and RDN catheter 114 are sized and configured for insertion into renal arteries (described in greater detail below). The imaging catheter 108 can be any of a variety of intraluminal imaging devices, such as, for example, an intravascular ultrasound (IVUS) catheter, an optical coherence tomography (OCT) catheter, or the like. Although the imaging catheter 108 could be implemented as an imaging catheter configured to acquire images via any of a variety of imaging modalities, a rotating ultrasound device, such as, an IVUS catheter, is used as an example in the balance of the disclosure (e.g., FIG. 2). However, this is not intended to be limiting.

Similarly, the RDN catheter 114 can be any of a variety of devices configured to emit energy (e.g., ablation energy, or the like) via any of a variety of modalities to target the renal nerves as part of an RDN procedure. For example, the RDN catheter 114 could be configured to emit RF energy, acoustic energy (e.g., ultrasound, or the like), or energy in the form of heat, etc. Although the RDN catheter 114 of FIG. 1 is agnostic to the type of ablation energy emitted by the RDN catheter 114, other examples (FIG. 3, etc.) describe an RDN catheter 114 using ultrasound transducers to emit acoustic energy. However, examples are not limited in this context.

During use, for example in an RDN procedure, the imaging catheter 108 can be inserted into a renal artery of a patient and indications of the intraluminal structure (e.g., plaque burden, tissue type, images, etc.) of the renal artery can be captured by the image acquisition console 106. These indications of the intraluminal structure can be communicated to the RDN subsystem 104. The RDN subsystem 104 can then use these indications to tailor (e.g., adjust, modulate, increase, decrease, pause, etc.) the ablation energy emitted during the RDN procedure. Examples of this are given in greater detail herein. However, prior to detailing the use of indications to tailor the ablation energy, more detailed examples of the image acquisition subsystem 102 and the RDN subsystem 104 are provided. In some instances, the imaging catheter 108 and the RDN catheter 114 may be inserted into the renal artery concurrently while in other instances, the imaging catheter 108 and the RDN catheter 114 can be inserted into the renal artery at separate times.

FIG. 2 illustrates an example image acquisition system 200, which could be implemented as the image acquisition subsystem 102 of the renal denervation (RDN) system 100 shown in FIG. 1. The image acquisition system 200 includes an image acquisition device 202, an IVUS catheter 204, a motor drive unit (MDU) 206, and an imaging subsystem 208. It is noted that this example uses an intraluminal imaging catheter configured to capture images via ultrasound. However, examples are not limited in this context.

The image acquisition device 202 is coupled to the IVUS catheter 204 via the MDU 206 and is also coupled to the imaging subsystem 208. In particular, the image acquisition device 202 is coupled to the MDU 206 via the MDU bus 210 while the MDU 206 is coupled to the IVUS catheter 204 via the catheter bus 212. In some embodiments, the MDU bus 210 and the catheter bus 212 can be transmission lines (or other conductors) arranged to convey signals between the connected components. For example, the MDU bus 210 and catheter bus 212 can be arranged to transmit voltage pulses (e.g., as control signals, as ultrasound pulse activation signals, as ultrasound echo return signals, or the like) between the various components of the image acquisition system 200. For example, the MDU bus 210 could be digital and/or serial busses, power transmission lines, etc.

In general, the image acquisition device 202 is configured to control the MDU 206 and receive signals from the IVUS catheter 204, via the MDU 206. Further, the image acquisition device 202 is configured to process the received signals to generate images and convey the images to the imaging subsystem 208. To that end, the image acquisition device 202 is coupled to the imaging subsystem 208 via the imaging subsystem bus 214, which can be a wired connection or a wireless connection. As a specific example, imaging subsystem bus 214 can be an Ethernet connection. In some examples, the imaging subsystem 208 can be a display, a tablet computer, or other device configured to display images rendered by image acquisition device 202. It is noted that although the imaging subsystem 208 is depicted external to image acquisition device 202, with some embodiments, imaging subsystem 208 can be incorporated into the same housing as image acquisition device 202.

The image acquisition device 202 includes an imaging processing circuitry 216, computer subsystem 218, and other subsystems 220. In general, the image processing circuitry 216 is configured to send and receive voltage pulses to the MDU 206 and IVUS catheter 204 while the computer subsystem 218 is configured with a processor 222 and memory 224. The memory 224 can store instructions and/or other data (e.g., images, indications of intraluminal structure, lesion mapping, or the like).

For example, the memory 224 can include instructions, which when executed by the processor 222 cause the computer subsystem 218 to cause the image acquisition device 202 to send and receive signals with the IVUS catheter 204 and motor drive unit (MDU) 206 to capture images from within a renal artery, as contemplated herein. The other subsystems 220 can include any of a variety of other systems or circuitry necessary for operation of the image acquisition device 202, such as, for example, power supply circuitry, control circuitry, etc.

Further, in some embodiments, the processor 222 can execute instructions stored in memory 224 to receive indications of structure in the renal arteries and to determine a location and/or type of structure. For example, the processor 222 can execute instructions stored in memory 224 to determine locations of lesions (e.g., plaque, calcium, etc.) along the renal arteries and communicate such information to an RDN subsystem (e.g., the RDN subsystem 104, or the like).

FIG. 3 illustrates an example RDN system 300, which could be implemented as the RDN subsystem 104 of the RDN system 100 shown in FIG. 1. The RDN system 300 includes an RDN console 302, an RDN catheter 304, and a motor drive unit (MDU) 306. The RDN console 302 is coupled to the RDN catheter 304 via the MDU 306. In particular, the RDN console 302 is coupled to the MDU 306 via the MDU bus 308 while the MDU 306 is coupled to the RDN catheter 304 via the catheter bus 310. It is noted that the MDU 306 is depicted separate from RDN console 302 in the example depicted in this figure. However, with some embodiments, the MDU 306 could be incorporated into the RDN console. As another example, an RDN system could be implemented with an omnidirectional RDN catheter 304. In such examples, the MDU 306 may not be necessary and may not be provided with such a system.

In some embodiments, the MDU bus 308 and the catheter bus 310 can be transmission lines (or other conductors) arranged to convey signals between the various components of the RDN system 300. For example, the MDU bus 308 and catheter bus 310 can be arranged to transmit activation signals (e.g., control signals, pulse activation signals, etc.) between the indicated components of the RDN system 300.

As noted above, an RDN system 300 can be implemented based on any of a variety of ablation energy modalities (e.g., RF, PEF, ultrasound, etc.) In the embodiment described in this figure, the RDN catheter 304 is configured to emit acoustic energy (e.g., via ultrasound emitters, or the like) where the acoustic energy is the ablation energy. However, it is noted that this is provided as an example only and is not intended to be limiting.

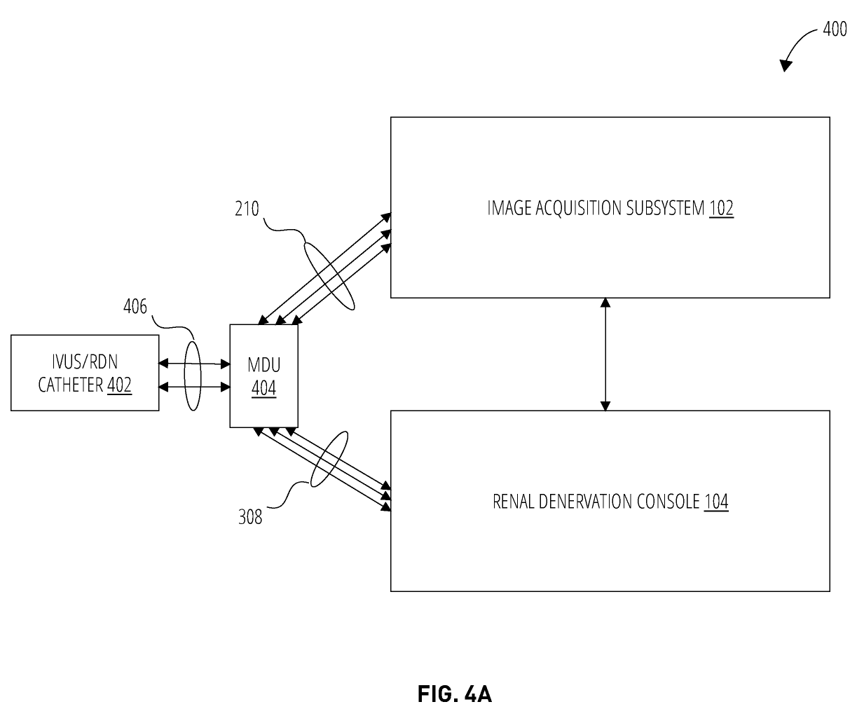

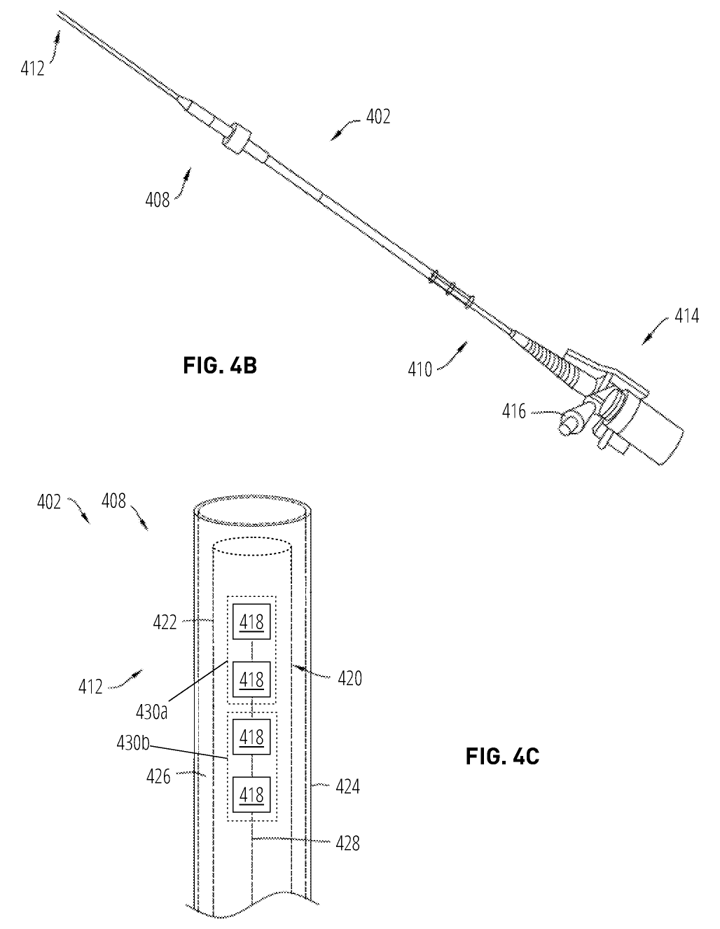

In general, the RDN console 302 is configured to control the MDU 306 and the RDN catheter 304. The RDN catheter 304 will be described in greater detail below. However, as a general introduction, the RDN catheter 304 comprises several transducers (e.g., see transducers 418 in FIG. 4B) configured to emit acoustic energy and arranged as an array of ultrasound transducers 312. The ultrasound transducers could be configured to be activated in a phased array. For example, the ultrasound transducers of the array of ultrasound transducers 312 can be selectively activated (e.g., with different activation delays and/or amplitudes) to steer, shape, and/or focus acoustic energy emitted from the ultrasound transducers 418 of the array of ultrasound transducers 312.

Additionally, the RDN console 302 can include a power supply 314, a generator 316, a controller 318, and a computer subsystem 320. In general, the power supply 314 can include power supply circuitry to convert line voltage (e.g., 120V alternating current, 240V alternating current, or the like) into voltage suitable for powering the other components of the RDN console 302. The generator 316 can include circuitry configured to generate activation signals, including voltage pulses to activate the ultrasound transducers 418 of the array of ultrasound transducers 312 as outlined herein. The controller 318 can include circuitry configured to control the generator 316 to cause the generator 316 to generate activation signals with varying activation delays for each of the ultrasound transducers 418 of the array of ultrasound transducers 312. Further, the controller 318 can control the generator to cause the generator to generate activation signals to cause the ultrasound transducers 418 to emit acoustic energy of a selected amplitude or different selected amplitudes. Additionally, the controller 318 can include circuitry to send signals to the MDU 306 to cause the MDU 306 to rotate the ultrasound transducers 418 as described in further detail below.

The computer subsystem 320 can include conventional computing structures, such as, processor 322 and memory 324. The memory 324 can store instructions and/or other data (e.g., target and/or non-target distances, an ablation volume, transducer pulse activation sequences for various distances and/or ablation volumes, or the like).

For example, the memory 324 can store instructions that when executed by the processor 322 cause the processor 322 to implement various functions. For example, the processor 322 can execute instructions stored in the memory 324 to receive indications of structure in a renal artery from an imaging subsystem (e.g., the image acquisition subsystem 102, the image acquisition system 200, or the like). Further, the processor 322 can execute instructions stored in the memory 324 to modulate (e.g., increase, reduce, pause, change, etc.) the ablation volume (e.g., power of acoustic energy, frequency of acoustic energy pulses, etc.) based on the indications.

With some embodiments, the controller 318 can be incorporated into the computer subsystem 320. For example, the processor 322 can execute instructions stored in the memory 324 to indicate to the generator 316 the activation pulse sequences to generate and send to the ultrasound transducers 418 of the array of ultrasound transducers 312 to cause the RDN catheter 304 to ablate renal nerve tissue based on indications of intraluminal structure.

Additionally, the computer subsystem 320 can include a display 326. With some embodiments, processor 322 can execute instructions stored in memory 324 to cause the display 326 to display various parameters of the renal denervation procedure and/or indications of the intraluminal structure.

FIG. 4A illustrates an example RDN system 400, which could be implemented as the RDN system 100 of FIG. 1. In general, the RDN system 400 includes the same components as the RDN system 100 with the exception that the imaging catheter 108 and RDN catheter 114 are combined into a single catheter. For example, RDN system 400 includes image acquisition console 106, RDN console 112, combined imaging and RDN catheter 402, and MDU 404.

With some embodiments, the image acquisition console 106 can be implemented by portions of the image acquisition system 200 of FIG. 2 while RDN console 112 can be implemented by portions of the RDN system 300 of FIG. 3. For example, image acquisition console 106 can be the image acquisition device 202 while RDN console 112 can be the RDN console 302.

The image acquisition console 106 and RDN console 112 are configured to couple to the combined imaging and RDN catheter 402 via the MDU 404. In general, the MDU 404 can be like the MDU 206 and/or MDU 306 with the exception that the MDU 404 is configured to communicate both imaging and RDN signals between the combined imaging and RDN catheter 402 and the image acquisition console 106 and RDN console 112.

The image acquisition console 106 is coupled to the MDU 404 via the MDU bus 210 while the RDN console 112 is coupled to the MDU 404 by the MDU bus 308. Further, the MDU 404 is coupled to the combined imaging and RDN catheter 402 via the catheter bus 406. The catheter bus 406 can be transmission lines (or other conductors) arranged to convey signals between the connected components of the RDN system 400. For example, the catheter bus 406 can be arranged to transmit activation signals (e.g., control signals, ultrasound emission signals, ultrasound echo return signals, etc.) between the indicated components of the RDN system 400.

The image acquisition console 106 and RDN conosle 112 can be interconnected to communicate signals and/or information elements (e.g., data structures, or the like). For example, image acquisition console 106 can communicate indications about the structure within the renal artery while the RDN console 112 can communicate indications about the ablation energy to be and/or being delivered. This is explained in greater detail below.

FIGS. 4B and 4C illustrate views of an example combined imaging and RDN catheter 402, according to at least one embodiment of the disclosure. FIGS. 4B and 4C illustrate side and perspective views of the RDN catheter 304 of the RDN system 300 of FIG. 3 in greater detail. As noted above, in some embodiments, the RDN system 100, image acquisition system 200, RDN system 300, or RDN system 400 described herein can be configured to operate with the combined imaging and RDN catheter 402.

The combined imaging and RDN catheter 402 includes an elongated member 408 having a proximal end 410 and a distal end 412. The combined imaging and RDN catheter 402 further includes a hub 414 coupled to the proximal end 410 of the elongated member 408 while the distal end 412 of the elongated member 408 is configured and arranged for percutaneous insertion into a patient (e.g., into renal arteries, or the like). In some instances, the elongated member 408 and the hub 414 are formed as a unitary body. In other instances, the elongated member 408 and the catheter hub 414 are formed separately and subsequently assembled.

The combined imaging and RDN catheter 402 further includes a driveshaft 420 disposed within the elongated member 408 and coupled to the hub 414 at proximal end of the drive shaft (not shown). As indicated above, the combined imaging and RDN catheter 402 includes ultrasound transducers 418 arranged in arrays 430a and 430b. With some embodiments, one of the arrays (e.g., array 430a, or the like) can be configured to emit and receive acoustic energy (e.g., ultrasound signals) to capture indications of structure in the renal arteries or indications of intraluminal images of the renal arteries; while another one of the arrays (e.g., array 430b, or the like) can be configured to emit acoustic energy to ablate and/or neuromodulate the renal nerves.

With some embodiments, the combined imaging and RDN catheter 402 may include a fluid port, such as flush port 416. The flush port 416 may be defined in the hub 414 and may be configured to flush and/or fill the elongated member 408 with fluid (e.g., saline, or the like). The hub 414 may be configured and arranged to couple to the MDU 404 of the RDN system 400. Mechanical energy from MDU 404 may be used to drive (or rotate) the driveshaft 420. As such, the ultrasound transducers 418 can rotate while emitting acoustic energy to capture indications of intraluminal structure or images and/or ablate renal tissue (e.g., renal nerves) in a circular or radial pattern about the longitudinal axis of the elongated member 408.

FIG. 4C is a perspective view of the distal end 412 of the elongated member 408 of the combined imaging and RDN catheter 402. With some embodiments, the elongated member 408 includes a sheath 424 with a longitudinal axis (e.g., a central longitudinal axis extending axially through the center of the sheath 424 and/or the combined imaging and RDN catheter 402) and a lumen 426. The driveshaft 420 is disposed in the lumen 426 of the sheath 424. As described, the ultrasound transducers 418 can be mounted to a distal end 422 of the driveshaft 420 and can be employed to transmit acoustic energy as part of an RDN procedure where the RDN procedure includes intraluminal interrogation as outlined herein.

The sheath 424 may be formed from any flexible, biocompatible material suitable for percutaneous insertion into a patient. Examples of suitable materials include, but are not limited to, polyethylene, polyurethane, plastic, spiral-cut stainless steel, nitinol hypotube, and the like or combinations thereof.

With some embodiments, the ultrasound transducers 418 are linearly disposed along the distal end 422 of the driveshaft 420. In some embodiments, the ultrasound transducers 418 are arranged in a rectangular pattern with multiple rows and columns array. For example, the ultrasound transducers 418 are depicted in FIG. 4C in a rectangular pattern with one (1) column and four (4) rows. Further, although ultrasound transducers 418 are depicted in a rectangular arrangement, the ultrasound transducers 418 could be arranged into other geometric arrangement, such as, an annular array, a square array, a circular array, or the like.

It is noted that the arrays 430a and 430b depicted in FIG. 4C only show four (4) ultrasound transducers 418 total. However, the combined imaging and RDN catheter 402 could be implemented with more or less than four (4) ultrasound transducers 418. In some embodiments, the combined imaging and RDN catheter 402 can be implemented with ten (10) ultrasound transducers 418, eleven (11) ultrasound transducers 418, or the like. Further, although not depicted in this figure, the arrays 430a and 430b need not have the same number of ultrasound transducers 418. For example, more ultrasound transducers 418 could be provided in an array configured to emit acoustic energy to ablate renal nerves (e.g., array 430a) than in an array configured to interrogate and/or image the intraluminal structure (e.g., array 430b).

Further, with some embodiments, the arrays may be non-exclusive. For example, array 430a may be configured to emit acoustic energy to ablate renal nerves and can comprise a group of ultrasound transducers 418 while array 430b can be configured to interrogate the intraluminal structure and can comprise another group of ultrasound transducers 418, where at least one of the ultrasound transducers 418 are members of both arrays 430a and 430b.

With some embodiments, the ultrasound transducers 418 are linearly spaced along the driveshaft 420 with a spacing of 0.2 mm (0.008 inches) to 2.0 mm (0.079 inches) between adjacent ultrasound transducers 418 or with a spacing of 0.5 mm (0.020 inches) to 5.0 mm (0.197 inches) between adjacent ultrasound transducers 418.

The ultrasound transducers 418 can be coupled to the image acquisition console 106 and RDN console 112 (via the MDU 404, or the like) with any number of conductors 428. The conductors 428 can electrically couple the ultrasound transducers 418 and catheter bus 406. In such a manner, voltage signals (e.g., ablation energy activation pulses, ultrasound interrogation pulses, ultrasound echo return pulses, or the like) can be transmitted from the image acquisition console 106 and/or RDN console 112 to the combined imaging and RDN catheter 402. In such an example, ones of the ultrasound transducers 418 could be tuned for imaging (e.g., configured to operate at 40 Megahertz (MHz), 60 MHz, or the like) while other ones of the ultrasound transducers 418 could be tuned for ablation (e.g., generally configured to operate at lower frequencies than the imaging transducers, such as, 5 MHz, 1 MHz, or the like).

Each of the ultrasound transducers 418 may be formed from materials capable of transforming applied electrical pulses to pressure distortions on the surface of the ultrasound transducers 418, and vice versa. In some embodiments, ones of the ultrasound transducers 418 may be simply ultrasound emitters and may not be capable of converting the acoustic pulses to electrical pulses while other ones of the ultrasound transducers 418 may be configured to both emit and receive acoustic pulses. It is noted that although the disclosure uses the ultrasound “transducers” throughout, examples are not to be limited in this respect and ones of the described ultrasound “transducers” could be ultrasound “emitters” or “receivers”.

Examples of suitable materials with which the ultrasound transducers 418 can be formed include piezoelectric ceramic materials, piezocomposite materials, piezoelectric plastics, barium titanates, lead zirconate titanates, lead metaniobates, polyvinylidene fluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like).

The pressure distortions on the surface of the ultrasound transducers 418 form acoustic pulses of a frequency based on the resonant frequencies of the ultrasound transducers 418. These acoustic pulses are emitted as acoustic energy. The resonant frequencies of the ultrasound transducers 418 may be affected by the size, shape, and material used to form the ultrasound transducers 418. The ultrasound transducers 418 may be formed in any shape suitable for positioning within the combined imaging and RDN catheter 402 and for propagating acoustic pulses of a desired frequency in one or more selected directions and for receiving echo return pulses. For example, the ultrasound transducers 418 may be disc-shaped, block-shaped, rectangular-shaped, oval-shaped, and the like. Further, the ultrasound transducers 418 may be formed in the desired shape by any process including, for example, dicing, dice and fill, machining, micro-fabrication, and the like.

As a specific example, each of the ultrasound transducers 418 may include a layer of piezoelectric material sandwiched between a matching layer and a conductive backing material formed from an acoustically absorbent material (e.g., an epoxy substrate with tungsten particles). During operation, the piezoelectric layer may be electrically excited to cause the emission of acoustic pulses.

The ultrasound transducers 418 can be used to emit acoustic energy towards tissue (e.g., renal artery tissue, or the like) and to receive echo return pulses responsive to the emitted acoustic energy as part of an intraluminal interrogation process. Accordingly, when the ultrasound transducers 418 are rotated about the longitudinal axis of the combined imaging and RDN catheter 402 and activated to emit acoustic energy, the tissue can be interrogated in a radial pattern about the longitudinal axis of the combined imaging and RDN catheter 402 such that indications of the intraluminal structure can be determined.

Further, the ultrasound transducers 418 can be used to apply acoustic energy to tissue (e.g., renal nerve tissue, or the like) to be ablated. Accordingly, when the ultrasound transducers 418 are rotated about the longitudinal axis of the combined imaging and RDN catheter 402 and activated to emit acoustic energy, tissue can be ablated in a radial pattern about the longitudinal axis of the combined imaging and RDN catheter 402.

As described above, acoustic energy emitted by the ultrasound transducers 418 can be modulated based on the indications of intraluminal structure.

With some embodiments, the ultrasound transducers 418 can be configured to operate within a frequency range of 5 MHz to 200 MHz

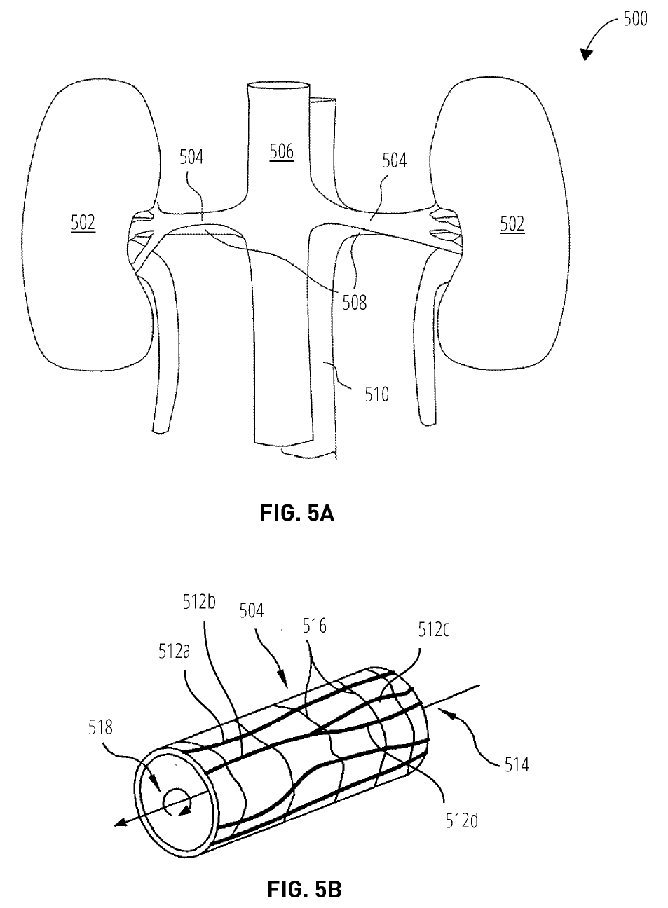

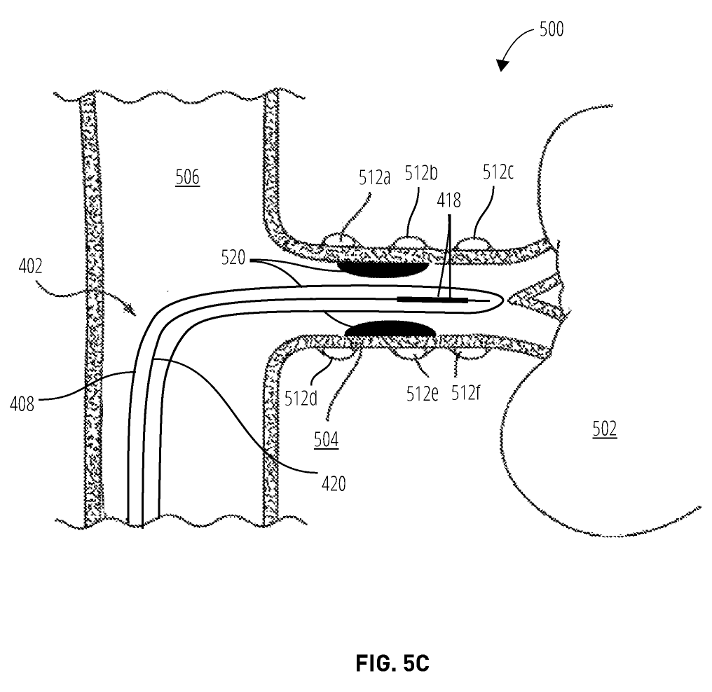

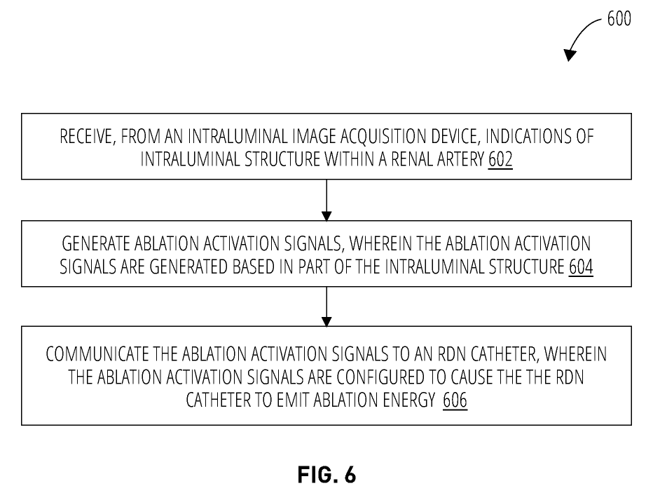

FIG. 5A illustrates a diagram depicting human renal anatomy 500, in which the various RDN procedures contemplated herein can be practiced. The human renal anatomy 500 includes kidneys 502, which are supplied with oxygenated blood by renal arteries 504. The renal arteries 504 are connected to the heart by the abdominal aorta 506. Deoxygenated blood flows from the kidneys 502 to the heart via renal veins 508 and the inferior vena cava 510.

FIG. 5B illustrates a portion of the human renal anatomy 500 of FIG. 5A in greater detail. More specifically, FIG. 5B depicts a portion of one of the renal arteries 504. As depicted, the renal artery 504 includes renal nerves 512a, 512b, 512c, and 512d, as well as renal nerves 512e and 512f (see FIG. 5C for renal nerves 512e and 512f) extending longitudinally along the longitudinal axis 514 of the renal artery 504, generally within the adventitia of the renal artery 504. Additionally, the renal artery 504 has smooth muscle cells 516 that surround the arterial circumference (e.g., around angular axis 518.

It is the renal nerves 512a to 512f that are the target for ablation during an RDN procedure. In general, the renal nerves 512a to 512f are between 3 mm (0.118 inches) and 5 mm (0.197 inches) from the renal artery 504.

FIG. 5C illustrates a portion of the human renal anatomy 500 with the combined imaging and RDN catheter 402 of the RDN system 400 from FIGS. 4A, 4B, and 4C inserted into the renal artery 504. As depicted, the elongated member 408 of the RDN catheter 304 is inserted into the renal artery 504. As discussed herein, the tissue of the renal artery 504 may include structure that can interfere with ablation energy emitted from device positioned within the renal artery 504. For example, the renal artery 504 is depicted with a lesion 520 (e.g., plaque, calcium, etc.) that can interfere with the propagation of acoustic energy from the combined imaging and RDN catheter 402 to the renal nerves 512a to 512f, which will affect the volume of ablation energy with which the target tissue (e.g., the renal nerves 512a to 512f) is exposed. Further, as noted above, preservation of the tissue of the renal artery 504 while ablating the adjacent nerve structure is desired.

With some embodiments, the combined imaging and RDN catheter 402 can be configured to be inserted into the renal artery 504 via a guide catheter (e.g., conventional guide catheter, pre-curved renal catheter, or the like), which is introduced via the common femoral artery or, alternatively, through a brachial/radial approach, and advanced to the renal artery under guidance (e.g., fluoroscopic imaging guidance).

Once inserted into the renal artery 504, the combined imaging and RDN catheter 402 can first be activated by interrogation activation signals (e.g., generated by computer subsystem 218 or other subsystems 220, or the like) to interrogate the intraluminal structure of the renal artery 504. For example, image acquisition console 106 can generate interrogation activation signals including voltage pulses configured to cause the MDU 404 to rotate the driveshaft 420 about the angular axis 518 while the ultrasound transducers 418 (or ones of the ultrasound transducers 418 of array 430a) are activated by the activation signals. As such, acoustic energy can be emitted from the ones of the ultrasound transducers 418 and ultrasound echo signals received by the ones of the ultrasound transducers 418. The image acquisition console 106 can be further configured to determine intraluminal structure of the renal artery 504 (e.g., identify the lesion 520, or the like) as discussed above.

Further, the image acquisition console 106 can be configured to communicate the indications of the intraluminal structure (e.g., indications of the lesion 520, or the like). With some embodiments, the indications of the lesion 520 can include indications of characteristics of the lesion 520 (e.g., type, size, thickness, location along the longitudinal axis 514, or the like).

Subsequently, the combined imaging and RDN catheter 402 can be again activated by ablation activation signals (e.g., generated by the generator 316, controller 318, and/or computer subsystem 320, or the like) to apply ablation energy to the renal artery 504. For example, image acquisition console 106 can generate ablation activation signals including voltage pulses configured to cause the MDU 404 to rotate the driveshaft 420 about the angular axis 518 while the ultrasound transducers 418 (or ones of the ultrasound transducers 418 of array 430b) are activated by the activation signals. As such, acoustic energy can be emitted from the ones of the ultrasound transducers 418 where the emitted acoustic energy is configured to neuromodulate the renal nerves 512a to 512f as discussed above. With another example, the combined imaging and RDN catheter 402 could be repositioned based on the indications of the lesion 520 (e.g., to areas with more favorable anatomy for renal denervation, or the like).

Of note, the ablation activation signals can be generated based in part on the indications of the intraluminal structure as discussed herein.

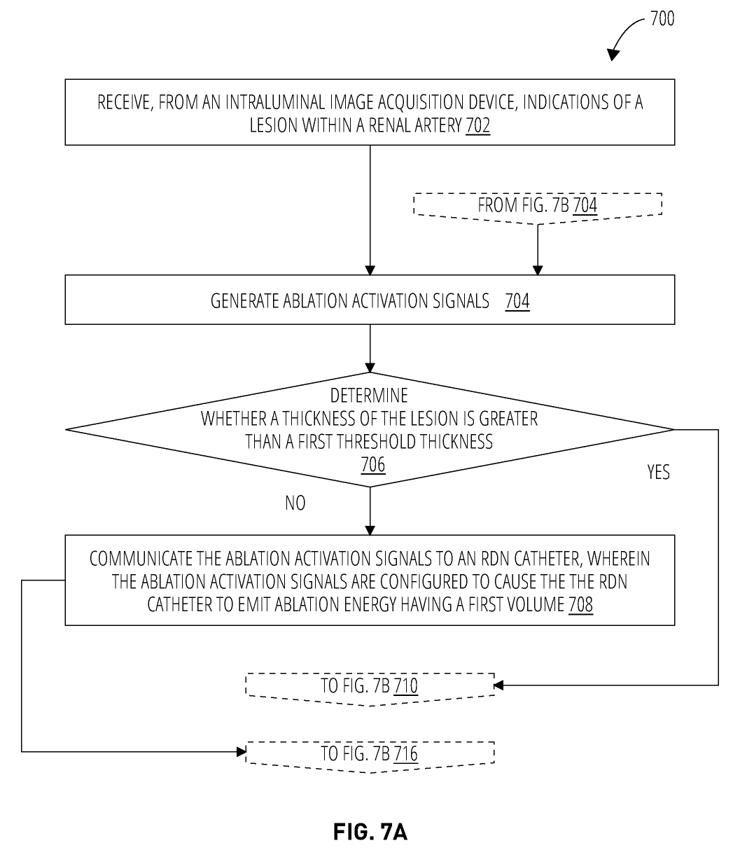

FIG. 6 illustrates a logic flow 600 to generate ablation activation signals based on indications of intraluminal structure, according to embodiments of the present disclosure. The logic flow 600 can be implemented by an RDN system, such as, the RDN system 100, the RDN system 300, or the RDN system 400. The RDN system 400 is used herein to describe the logic flow 600. However, it is noted that logic flow 600 could also be implemented by an RDN system different than that described herein.

Logic flow 600 can begin at block 602. At block 602 “receive, from an intraluminal image acquisition device, indications of intraluminal structure within a renal artery” indications of intraluminal structure of a renal artery can be received from an intraluminal image acquisition device. For example, processor 322 of RDN console 112 can execute instructions stored in memory 324 to cause the RDN console 112 to receive from image acquisition subsystem 102, indications of intraluminal structure within a renal artery. As a specific example, processor 322 of RDN console 112 can execute instructions stored in memory 324 to cause the RDN console 112 to receive from image acquisition subsystem 102, indications of lesion 520 of the renal artery 504 in which the combined imaging and RDN catheter 402 is inserted.

Continuing to block 604 “generate ablation activation signals, wherein the ablation activation signals are generated based in part of the intraluminal structure” ablation activation signals can be generated based on the intraluminal structure. For example, processor 322 of RDN console 112 can execute instructions stored in memory 324 to cause the RDN console 112 to generate (e.g., via generator 316, or the like) ablation activation signals based on the received indications of intraluminal structure. As a specific example, processor 322 of RDN console 112 can execute instructions stored in memory 324 to cause the RDN console 112 to generate ablation activation signals based on the indication of lesion 520 (e.g., based on the type, size, location etc.)

Continuing to block 606 “communicate the ablation activation signals to an RDN catheter, wherein the ablation activation signals are configured to cause the RDN catheter to emit ablation energy” the ablation activation signals can be communicated to an RDN catheter to cause the RDN catheter to emit energy (e.g., RF, PEF, ultrasound, or the like) to ablate, or neuromodulate, renal nerves of the renal artery. For example, processor 322 of RDN console 112 can execute instructions stored in memory 324 to cause the RDN console 112 to communicate the ablation activation signals to the MDU 404 and combined imaging and RDN catheter 402 such that the combined imaging and RDN catheter 402 emits acoustic energy from ones of the ultrasound transducers 418.

With some embodiments, logic flow 600 can be paired with an intraluminal interrogation process. For example, logic flow 600 can include, or follow, a logic flow to cause an imaging catheter (e.g., combined imaging and RDN catheter 402, or the like) to capture images from within the renal artery 504. The indications of intraluminal structure can then be generated and/or identified from the images.

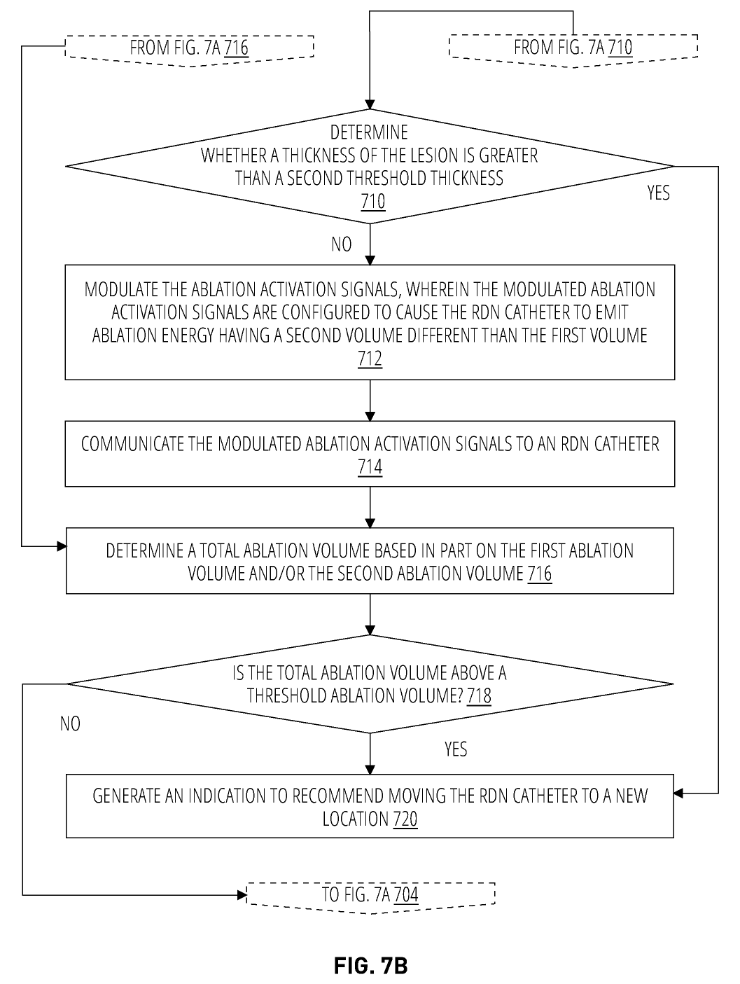

FIGS. 7A and 7B illustrate a logic flow 700 to generate ablation activation signals and modulate the generated ablation activation signals based on indications of intraluminal structure, according to embodiments of the present disclosure. Like logic flow 600, the logic flow 700 can be implemented by an RDN system, such as, the RDN system 100, the RDN system 300, or the RDN system 400. The RDN system 400 is used herein to describe the logic flow 700. However, it is noted that logic flow 700 could also be implemented by an RDN system different than that described herein.

Starting with FIG. 7A, logic flow 700 can begin at block 702 At block 702 “receive, from an intraluminal image acquisition device, indications of a lesion within a renal artery” indications of a lesion within a renal artery can be received. For example, processor 322 of RDN console 112 can execute instructions stored in memory 324 to cause the RDN console 112 to receive (e.g., from image acquisition subsystem 102) indications of lesion 520 within renal artery 504.

Continuing to block 704 “generate ablation activation signals” ablation activation signals can be generated. For example, processor 322 of RDN console 112 can execute instructions stored in memory 324 to cause the RDN console 112 to generate (e.g., via generator 316, or the like) ablation activation signals. Continuing to decision block 706 “determine whether a thickness of the lesion is greater than a first threshold thickness” a determination of whether the thickness of the lesion is greater than (or greater than or equal to) a first thickness threshold can be made. For example, processor 322 of RDN console 112 can execute instructions stored in memory 324 to cause the RDN console 112 to determine whether a thickness of the lesion 520 is greater than (or greater than or equal to) a thickness threshold. With some embodiments, the thickness threshold can be 0.3 mm (0.012 inches). With some embodiments, the thickness threshold can be 0.5 mm. With some embodiments, the thickness threshold can be between 0.5mm and 1 mm (0.039 inches).

From decision block 706, logic flow 700 can continue to either block 708 or decision block 710. In particular logic flow 700 can continue from decision block 706 to block 708 based on a determination at decision block 706 that the thickness of the lesion is not greater than (or greater than or equal to) the first thickness threshold while logic flow 700 can continue from decision block 706 to decision block 710 based on a determination at decision block 706 that the thickness of the lesion is greater than (or greater than or equal to) the first thickness threshold.

At block 708 “communicate the ablation activation signals to an RDN catheter, wherein the ablation activation signals are configured to cause the RDN catheter to emit ablation energy having a first volume” the ablation activation signals can be communicated to an RDN catheter to cause the RDN catheter to emit ablation energy (e.g., RF, PEF, ultrasound, or the like) having a first ablation volume to ablate, or neuromodulate, renal nerves of the renal artery. For example, processor 322 of RDN console 112 can execute instructions stored in memory 324 to cause the RDN console 112 to communicate the ablation activation signals to the MDU 404 and combined imaging and RDN catheter 402 such that the combined imaging and RDN catheter 402 emits acoustic energy from ones of the ultrasound transducers 418 having a first ablation volume. As used here, the term “ablation volume” can indicate any one or more characteristics of the emitted ablation energy (e.g., power, frequency, pulse duration, etc.) From block 708, logic flow 700 can continue to block 720.

Referring now to FIG. 7B, at decision block 710 “determine whether a thickness of the lesion is greater than a second threshold thickness” a determination of whether the thickness of the lesion is greater than (or greater than or equal to) a second thickness threshold can be made. For example, processor 322 of RDN console 112 can execute instructions stored in memory 324 to cause the RDN console 112 to determine whether a thickness of the lesion 520 is greater than (or greater than or equal to) a second thickness threshold, where the second thickness threshold is greater than the first thickness threshold. With some embodiments, the thickness threshold can be 0.3 millimeters (mm). With some embodiments, the thickness threshold can be 0.5 mm. With some embodiments, the thickness threshold can be between 0.5mm and 1 mm.

From decision block 710, logic flow 700 can continue to either block 712 or block 720. In particular logic flow 700 can continue from decision block 710 to block 712 based on a determination at decision block 710 that the thickness of the lesion is not greater than (or greater than or equal to) the second thickness threshold while logic flow 700 can continue from decision block 710 to block 720 based on a determination at decision block 710 that the thickness of the lesion is greater than (or greater than or equal to) the second thickness threshold.

At block 712 “modulate the ablation activation signals, wherein the modulated ablation activation signals are configured to cause the RDN catheter to emit ablation energy having a second volume different than the first volume” the ablation activation signals can be modulated such that the modulated ablation activation signals are configured to cause the RDN catheter to emit ablation energy having a second volume different than the first volume. For example, processor 322 of RDN console 112 can execute instructions stored in memory 324 to cause the RDN console 112 to modulate the ablation activation signals such that ablation energy emitted based on the activation signals has a different ablation volume than the first ablation volume. As a specific example, processor 322 of RDN console 112 can execute instructions stored in memory 324 to cause the RDN console 112 to modulate the ablation activation signals such that emitted ablation energy has a different power than the first ablation volume.

With some embodiments, the ablation activation signals can be modulated based on the lesion 520, or based on indications of the lesion 520 (e.g., the lesion type, etc.)

Logic flow 700 can continue from block 712 to block 714. At block 714 “communicate the modulated ablation activation signals to an RDN catheter” the modulated ablation activation signals can be communicated to the RDN catheter. For example, processor 322 of RDN console 112 can execute instructions stored in memory 324 to cause the RDN console 112 to communicate the modulated ablation activation signals to the MDU 404 and combined imaging and RDN catheter 402 such that the combined imaging and RDN catheter 402 emits acoustic energy from ones of the ultrasound transducers 418 having a second ablation volume.

Continuing to block 716 “determine a total ablation volume based in part on the first ablation volume and/or the second ablation volume” a total ablation volume delivered at the current location can be determined. For example, processor 322 of RDN console 112 can execute instructions stored in memory 324 to cause the RDN console 112 to determine the ablation volume delivered at the current location (e.g., based in part on the first ablation volume and/or the second ablation volume delivered at block 708 and/or 714). As used herein, the total ablation volume can be characterized at the total ablation energy emitted by the transducer(s) at a particular location.

Continuing to decision block 718 “is the total ablation volume above a threshold ablation volume?” a determination can be made of whether the total ablation volume is greater than (or greater than or equal to) a threshold ablation volume. For example, processor 322 of RDN console 112 can execute instructions stored in memory 324 to cause the RDN console 112 to determine whether the total ablation volume is greater than (or greater than or equal to) an ablation volume threshold.

From decision block 718, logic flow 700 can return to block 704 or continue to block 720. In particular logic flow 700 can return to block 704 from decision block 718 based on a determination at decision block 718 that the total ablation volume is not greater than (or greater than or equal to) the ablation volume threshold while logic flow 700 can continue from decision block 718 to block 720 based on a determination at decision block 718 that the total ablation volume is greater than (or greater than or equal to) the ablation volume threshold.

At block 720 “generate an indication to recommend moving the RDN catheter to a new location” an indication recommending moving the RDN catheter to a new location can be generated. For example, processor 322 of RDN console 112 can execute instructions stored in memory 324 to cause the RDN console 112 to generate an indication (e.g., graphical indication on a graphical user interface (GUI), audible indication, visual indication, or the like) where the indication is to provide a recommendation to move the RDN catheter 114 to a new location. From block 720, logic flow 700 can return to block 704.

With some embodiments, a mapping of structure (e.g., lesions 520, or the like) within the renal artery 504 can be generated and the ablation activation signals can be generated and modulated based on the mapping. For example, the location of the lesion 520 along the longitudinal axis 514 can be mapped such that the ablation activation signals can be modulated while ablation energy is being emitted in the location. As a specific example, the location of the lesion 520 can be mapped such that when ultrasound transducers 418 of the combined imaging and RDN catheter 402 are at the mapped locations, the ablation activation signals can be modulated such that the emitted acoustic energy is adjusted to account for the lesion.

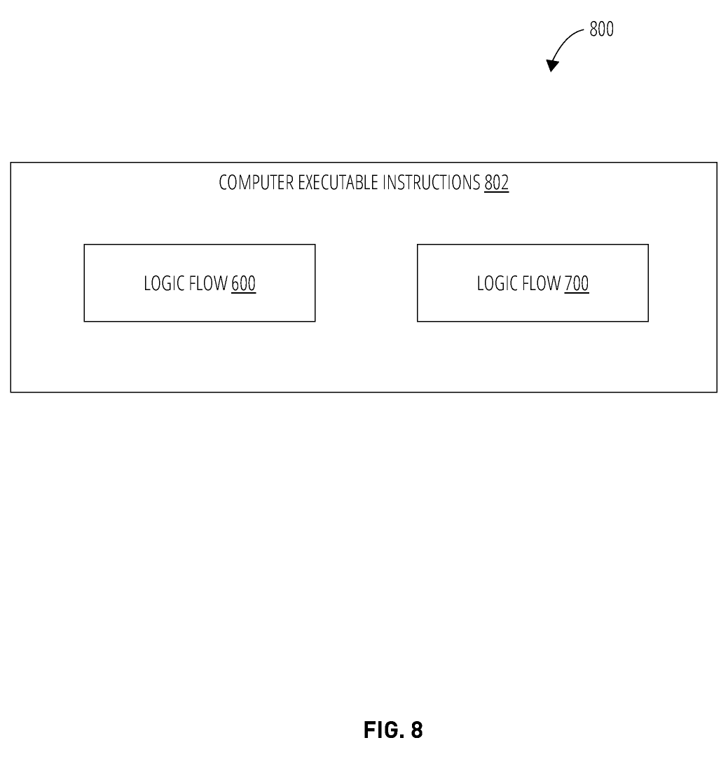

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., processor 222, processor 322, or the like) can execute. For example, computer executable instructions 802 can include instructions to implement operations described with respect to logic flow 600 and/or logic flow 700. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

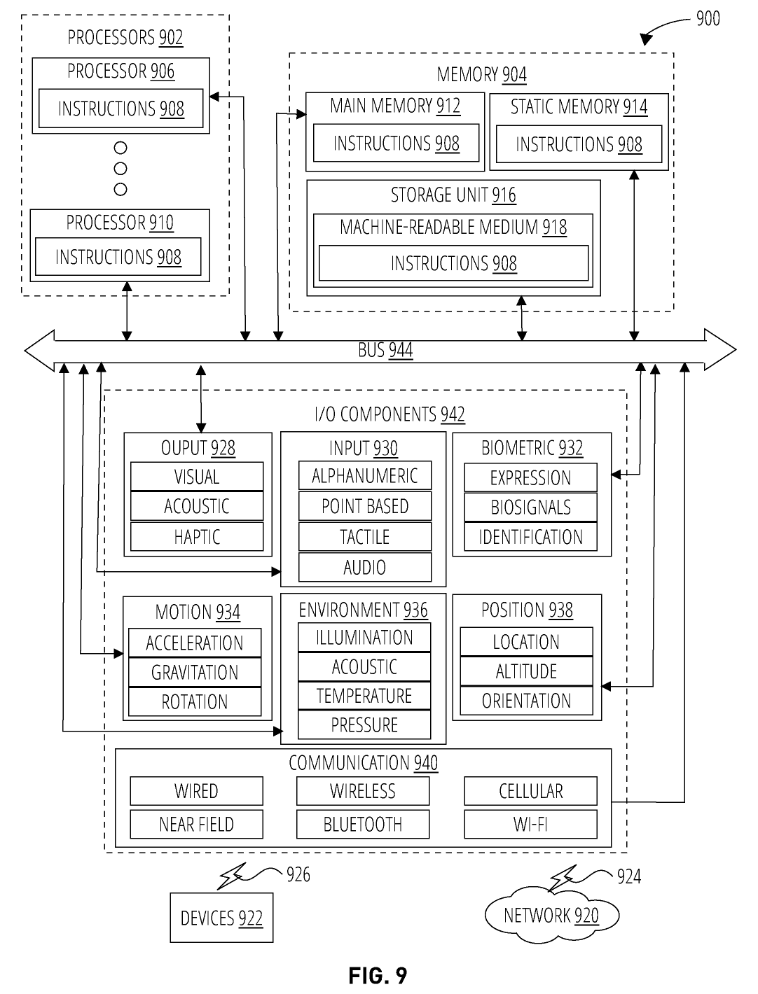

FIG. 9 illustrates a diagrammatic representation of a machine 900 in the form of a computer system within which a set of instructions may be executed for causing the machine to perform any one or more of the methodologies discussed herein. More specifically, FIG. 9 shows a diagrammatic representation of the machine 900 in the example form of a computer system, within which instructions 908 (e.g., software, a program, an application, an applet, an app, or other executable code) for causing the machine 900 to perform any one or more of the methodologies discussed herein may be executed. For example, the instructions 908 may cause the machine 900 to execute logic flow 600 of FIG. 6, logic flow 700 of FIG. 7, or the like. More generally, the instructions 908 may cause the machine 900 to receive indication of intraluminal structure, generate a mapping of the intraluminal structure, and/or modulate ablation energy emitted from an RDN catheter based on the indications and/or mapping.

The instructions 908 transform the general, non-programmed machine 900 into a particular machine 900 programmed to carry out the described and illustrated functions in a specific manner. In alternative embodiments, the machine 900 operates as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 908, sequentially or otherwise, that specify actions to be taken by the machine 900. Further, while only a single machine 900 is illustrated, the term “machine” shall also be taken to include a collection of machines 200 that individually or jointly execute the instructions 908 to perform any one or more of the methodologies discussed herein.

The machine 900 may include processors 902, memory 904, and I/O components 942, which may be configured to communicate with each other such as via a bus 944. In an example embodiment, the processors 902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 906 and a processor 910 that may execute the instructions 908. The term “processor” is intended to include multi-core processors that may comprise two or more independent processors (sometimes referred to as “cores”) that may execute instructions contemporaneously. Although FIG. 9 shows multiple processors 902, the machine 900 may include a single processor with a single core, a single processor with multiple cores (e.g., a multi-core processor), multiple processors with a single core, multiple processors with multiples cores, or any combination thereof.

The memory 904 may include a main memory 912, a static memory 914, and a storage unit 916, both accessible to the processors 902 such as via the bus 944. The main memory 904, the static memory 914, and storage unit 916 store the instructions 908 embodying any one or more of the methodologies or functions described herein. The instructions 908 may also reside, completely or partially, within the main memory 912, within the static memory 914, within machine-readable medium 918 within the storage unit 916, within at least one of the processors 902 (e.g., within the processor’s cache memory), or any suitable combination thereof, during execution thereof by the machine 900.

The I/O components 942 may include a wide variety of components to receive input, provide output, produce output, transmit information, exchange information, capture measurements, and so on. The specific I/O components 942 that are included in a particular machine will depend on the type of machine. For example, portable machines such as mobile phones will likely include a touch input device or other such input mechanisms, while a headless server machine will likely not include such a touch input device. It will be appreciated that the I/O components 942 may include many other components that are not shown in FIG. 9. The I/O components 942 are grouped according to functionality merely for simplifying the following discussion and the grouping is in no way limiting. In various example embodiments, the I/O components 942 may include output components 928 and input components 930. The output components 928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 942 may include biometric components 932, motion components 934, environmental components 936, or position components 938, among a wide array of other components. For example, the biometric components 932 may include components to detect expressions (e.g., hand expressions, facial expressions, vocal expressions, body gestures, or eye tracking), measure bio signals (e.g., blood pressure, heart rate, body temperature, perspiration, or brain waves), identify a person (e.g., voice identification, retinal identification, facial identification, fingerprint identification, or electroencephalogram-based identification), and the like. The motion components 934 may include acceleration sensor components (e.g., accelerometer), gravitation sensor components, rotation sensor components (e.g., gyroscope), and so forth. The environmental components 936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 942 may include communication components 940 operable to couple the machine 900 to a network 920 or devices 922 via a coupling 924 and a coupling 926, respectively. For example, the communication components 940 may include a network interface component or another suitable device to interface with the network 920. In further examples, the communication components 940 may include wired communication components, wireless communication components, cellular communication components, Near Field Communication (NFC) components, Bluetooth^{®} components (e.g., Bluetooth^{®} Low Energy), Wi-Fi^{®} components, and other communication components to provide communication via other modalities. The devices 922 may be another machine or any of a wide variety of peripheral devices (e.g., a peripheral device coupled via a USB).

Moreover, the communication components 940 may detect identifiers or include components operable to detect identifiers. For example, the communication components 940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 940, such as location via Internet Protocol (IP) geolocation, location via Wi-Fi® signal triangulation, location via detecting an NFC beacon signal that may indicate a particular location, and so forth.

The various memories (i.e., memory 904, main memory 912, static memory 914, and/or memory of the processors 902) and/or storage unit 916 may store one or more sets of instructions and data structures (e.g., software) embodying or utilized by any one or more of the methodologies or functions described herein. These instructions (e.g., the instructions 908), when executed by processors 902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 920 or a portion of the network 920 may include a wireless or cellular network, and the coupling 924 may be a Code Division Multiple Access (CDMA) connection, a Global System for Mobile communications (GSM) connection, or another type of cellular or wireless coupling. In this example, the coupling 924 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 908 may be transmitted or received over the network 920 using a transmission medium via a network interface device (e.g., a network interface component included in the communication components 940) and utilizing any one of several well-known transfer protocols (e.g., hypertext transfer protocol (HTTP)). Similarly, the instructions 908 may be transmitted or received using a transmission medium via the coupling 926 (e.g., a peer-to-peer coupling) to the devices 922. The terms “transmission medium” and “signal medium” mean the same thing and may be used interchangeably in this disclosure. The terms “transmission medium” and “signal medium” shall be taken to include any intangible medium that can store, encoding, or carrying the instructions 908 for execution by the machine 900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A renal denervation (RDN) system, comprising:
a generator configured to generate ablation activation signals to cause an RDN catheter to emit ablation energy;
a processor coupled to the generator; and
a memory comprising instructions, which when executed by the processor cause the processor to:
receive, from an image acquisition device, indications of intraluminal structure;
generate, via the generator, the ablation activation signals based on the indications of the intraluminal structure; and
communicate the ablation activation signals to the RDN catheter.

2. The RDN system of claim 1, comprising the RDN catheter.

3. The RDN system of claim 2, wherein the RDN catheter comprises:
an elongate member;
a drive shaft disposed within the elongate member; and
a plurality of ultrasound transducers disposed on a distal end of the drive shaft and configured to emit the ablation energy in the form of acoustic energy.

4. The RDN system of claim 3, wherein the plurality of ultrasound transducers are spaced linearly along the driveshaft.

5. The RDN system of claim 3, wherein each of the plurality of ultrasound transducers are spaced a distance from adjacent ones of the plurality of ultrasound transducers, and wherein the distance is between 0.2 millimeters (mm) and 5.0 mm or wherein the distance is between 0.5 mm and 2.0 mm.

6. The RDN system of claim 3, wherein the plurality of ultrasound transducers comprise between one (1) and eleven (11). ultrasound transducers.

7. The RDN system of claim 6, wherein the generator is a first generator, the processor is a first processor, and the memory is a first memory, and wherein the image acquisition device comprises:
a second generator configured to generate image activation signals to cause an imaging catheter to interrogate a renal artery;
a second processor coupled to the second generator; and
a second memory comprising instructions, which when executed by the second processor cause the second processor to:
generate, via the second generator, the image activation signals;
communicate the image activation signals to the imaging catheter;
receive, from the imaging catheter, the indications of the intraluminal structure; and
communicate the indications of the intraluminal structure to the first processor.

8. The RDN system of claim 6, wherein the imaging catheter and the RDN catheter are the same catheter.

9. The RDN of claim 6, comprising a motor drive unit (MDU) coupled between the generator and the imaging catheter and/or the RDN catheter.

10. The RDN system of claim 1, the memory further comprising instructions that when executed by the processor cause the processor to modulate the ablation activation signals based on the indications of intraluminal structure, wherein the modulated ablation activation signals cause the RDN catheter to emit modulated ablation energy.

11. The RDN system of claim 1, the memory further comprising instructions that when executed by the processor cause the processor to:
generate a mapping of the intraluminal structure from the indications of the intraluminal structure; and
modulate the ablation activation signals based on the mapping of the intraluminal structure,
wherein the modulated ablation activation signals cause the RDN catheter to emit modulated ablation energy.

12. The RDN system of claim 11, wherein the indications of intraluminal structure comprise indications of a lesion.

13. The RDN system of claim 12, wherein the mapping comprises an indication of a location of the lesion along a longitudinal axis of the renal artery and wherein the ablation activation signals are modulated at the locations.

14. The RDN system of claim 11, wherein the modulated ablation energy has a different power, different frequency, and/or a different pulse delay than the ablation energy.

15. A renal denervation (RDN) method, comprising:
providing an image acquisition device and an RDN device;
positioning the image acquisition device and the RDN device in a location in a renal artery of a patient; and
exposing, via the RDN device, the location in the renal artery to ablation energy to cause renal denervation of the renal nerves adjacent to the location in the renal artery,
wherein the ablation energy is generated by a generator coupled to the image acquisition device and the RDN device, and
wherein the ablation energy is generated based in part on indications, received from the image acquisition device, of intraluminal structure of the location in the renal artery.

16. The RDN method of claim 15, wherein the image acquisition device and the RDN device are incorporated in an intravenous catheter.

17. The RDN method of claim 16, wherein the comprising:
moving the intravenous catheter to a secondary location in the renal artery; and
exposing, via the RDN device, the secondary location in the renal artery to secondary ablation energy to cause renal denervation of the renal nerves adjacent to the secondary location in the renal artery,
wherein the secondary ablation energy is generated based in part on indications, received from the image acquisition device, of intraluminal structure of the secondary location in the renal artery, and
wherein the secondary ablation energy is different from the ablation energy.

18. A computer-readable storage device, storing instructions that when executed by a processor of a renal denervation (RDN) system, cause the RDN system to:
receive, from an image acquisition device coupled to the RDN system, indications of intraluminal structure of a renal artery;
generate, via a generator of the RDN system, ablation activation signals based on the indications of the intraluminal structure; and
communicate the ablation activation signals to an RDN catheter coupled to the RDN system, wherein the RDN catheter is configured to emit ablation energy responsive to the ablation activation signals.

19. The computer-readable storage device of claim 18, the instructions when executed by the processor of the RDN system, further cause the RDN system to:
modulate the ablation activation signals based on the indications of intraluminal structure,
wherein the modulated ablation activation signals cause the RDN catheter to emit modulated ablation energy.

20. The computer-readable storage device of claim 18, the instructions when executed by the processor of the RDN system, further cause the RDN system to:
generate a mapping of the intraluminal structure from the indications of the intraluminal structure; and
modulate the ablation activation signals based on the mapping of the intraluminal structure,
wherein the modulated ablation activation signals cause the RDN catheter to emit modulated ablation energy.
